(19) United States
(12) Patent Application Publication
(10) Pub. No.: US 2014/0200421 A1
(43) Pub. Date: Jul. 17, 2014

(54) PHYSIOLOGICAL SENSOR HAVING BIODEGRADABLE OPTICS

(71) Applicant: COVIDIEN LP, Mansfield, MA (US)

(72) Inventor: Bruce R. Gilland, Superior, CO (US)

(73) Assignee: COVIDIEN LP, Mansfield, MA (US)

(21) Appl. No.: 13/741,140

(22) Filed: Jan. 14, 2013

(51) Int. Cl. A61B 5/1459; A61B 5/00; A61B 18/12; A61B 18/18; A61B 5/1455; A61B 5/0205

(52) U.S. Cl. CPC A61B 5/1459; A61B 5/14552; A61B 5/0205; A61B 18/12; A61B 18/1815; A61B 5/4836
USPC 600/325; 600/478

(57) ABSTRACT

Medical sensor assemblies configured to provide enhanced patient comfort when worn over a period of time are provided. The medical sensor assemblies may include sensors in optical communication with one or more biodegradable light guides that facilitate the transmission of light to and from an internal tissue of the patient. The medical sensor assemblies may also include a bandage that facilitates coupling of the light guides to the sensor. Additionally or alternatively, a bandage may be positioned against an internal tissue to specifically direct the emitted light through the tissue. Such embodiments may provide enhanced light transmission between the emitter and detector of the sensors.

### BACKGROUND

The present disclosure relates generally to patient monitoring systems and, more particularly, to patient sensors having biodegradable features for non-invasive and invasive physiological monitoring.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Patient sensors are used in a variety of medical applications to determine physiological parameters of a patient. For example, a pulse oximetry sensor may perform measurements such that a patient's pulse rate and/or blood oxygen saturation may be determined. Such patient sensors may communicate with a patient monitor using a communication cable. For example, a patient sensor may use such a communication cable to send a signal, corresponding to a measurement performed by the sensor, to the patient monitor for processing. However, the use of communication cables may limit the range of applications available, as the cables may become prohibitively expensive at long distances as well as limit a patient's range of motion by physically tethering the patient to a monitoring device.

In addition, in certain circumstances, it may be desirable to monitor the physiological parameters of the patient over time. For example, hospital stays of one or more days after a surgical procedure can be common, and the monitoring performed during this stay can be intrusive and uncomfortable for the patient. While certain monitors, such as pulse oximetry monitors, may be equipped with features (e.g., wireless communication technologies) that enable a patient to freely move about while monitoring is being performed, wireless patient sensors can oftentimes be bulky. For example, in such wireless sensors, a large portion of the bulk and weight of the sensor may be attributable to the battery used to power the sensor. Therefore, enhanced comfort for a wireless sensor could potentially be achieved if the power requirements and the battery size of the sensor could be reduced.

### BRIEF DESCRIPTION OF THE DRAWINGS

Advantages of the disclosed techniques may become apparent upon reading the following detailed description and upon reference to the drawings in which:

FIG. 1 is a perspective view of a patient sensor system having a wireless patient sensor incorporating biodegradable optics, in accordance with an embodiment;

FIG. 2 is a block diagram of the wireless patient sensor of FIG. 1, in which the wireless patient sensor is in communication with a bandage having biodegradable light guides inserted into a patient tissue, in accordance with an embodiment;

FIG. 3 is an expanded view of an optical lens of the wireless patient sensor of FIG. 1 having biodegradable microneedles, in accordance with an embodiment;

FIG. 4 is an exploded view of a bandage having biodegradable optics configured to insert into a patient tissue, and a patient sensor capable of working in conjunction with the bandage, in accordance with an embodiment;

FIG. 5 is a cross-sectional side view of the bandage and patient sensor of FIG. 4, where the biodegradable optics have been inserted into a patient tissue for invasive or semi-invasive physiological monitoring, in accordance with an embodiment;

FIG. 6 is a flowchart illustrating a process for positioning the bandage and patient sensor of FIG. 4 to enable invasive or semi-invasive physiological monitoring, in accordance with an embodiment;

FIG. 7 is a cross-sectional side view of a hypodermic needle being utilized to insert the biodegradable optics of the bandage into a patient for invasive monitoring according to the process of FIG. 6, in accordance with an embodiment;

FIG. 8 is a cross-sectional side view of the biodegradable optics of the bandage after insertion into the patient and after the needle of FIG. 7 has been retracted, in accordance with an embodiment;

FIG. 9 is a cross-sectional side view of the biodegradable optics of the bandage after coupling to the bandage of FIG. 4, in accordance with an embodiment;

FIG. 10 is a cross-sectional side view of an internal-use bandage coupled to a patient tissue and also communicatively coupled to the bandage and patient sensor of FIG. 4, in accordance with an embodiment;

FIG. 11 is a flowchart illustrating a process for positioning biodegradable optics relative to an internal tissue to enable invasive physiological monitoring, in accordance with an embodiment;

FIG. 12 is a flowchart illustrating a process for using biodegradable optics and a patient sensor for monitoring a post-operative tissue, in accordance with an embodiment;

FIG. 13 is a diagrammatical illustration of a patient sensor being used to perform invasive monitoring of a post-surgical tissue, in accordance with an embodiment;

FIG. 14 is a diagrammatical illustration of a surgical system having biodegradable optics enabling a patient sensor to monitor an internal tissue during a surgical procedure, in accordance with an embodiment; and

FIG. 15 is a flowchart of a process for using biodegradable optics for invasive monitoring of a tissue during a surgical procedure, and adjusting the surgical procedure based on the monitoring, in accordance with an embodiment.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As noted above, wireless patient sensors may be used to afford a patient a greater freedom of movement compared to wired patient sensors. However, these wireless sensors can be bulky, which can be uncomfortable for the patient. In addition, some monitoring techniques performed during extended hospital stays (e.g., after a surgical procedure) may not necessarily provide information directly related to post-operative tissue. Rather, many monitoring techniques simply provide physiological parameter data that is averaged over a region of the body. Therefore, in some situations, imaging techniques such as ultrasound imaging, magnetic resonance imaging, and/or X-ray/computed tomography imaging, may be used to assess the condition of post-operative tissue. Alternatively, the patient may simply wait to determine whether the tissue is healing/has healed based on restored function or another subjective factor.

To address these and other shortcomings of existing approaches, the present disclosure provides embodiments in which biodegradable components, such as biodegradable light guides, are used in conjunction with one or more non-invasive patient sensors to monitor physiological parameters of a particular tissue, such as a pre-operative, post-operative, or intra-operative tissue. For example, the biodegradable light guides may be attached to, or otherwise in communication with, one or more optical components of a sensor (e.g., a light emitter and/or light detector). The biodegradable light guides may also be attached or inserted into an internal patient tissue for direct monitoring of the tissue using the sensor. Therefore, such embodiments may enable the monitoring of deep tissues using only mildly invasive features. Over time, the biodegradable light guides may be absorbed by the body, obviating the need for later removal from the patient, and also mitigating the risk of subsequent infection or other complications.

In addition, biodegradable light guides in accordance with the present disclosure may enable more precise delivery of certain wavelengths of light used for monitoring (e.g., visible light such as red light, infrared (IR) light) into perfuse tissue compared to typical sensor configurations. This more precise delivery in turn may reduce the amount (e.g., flux) of light used for monitoring, which also reduces power use. Therefore, the present embodiments may enable reduced power consumption by wireless patient sensors, which are battery-powered. The reduced power consumption by the optics can result in longer operational time between charges and reduced battery size, among other enhancements.

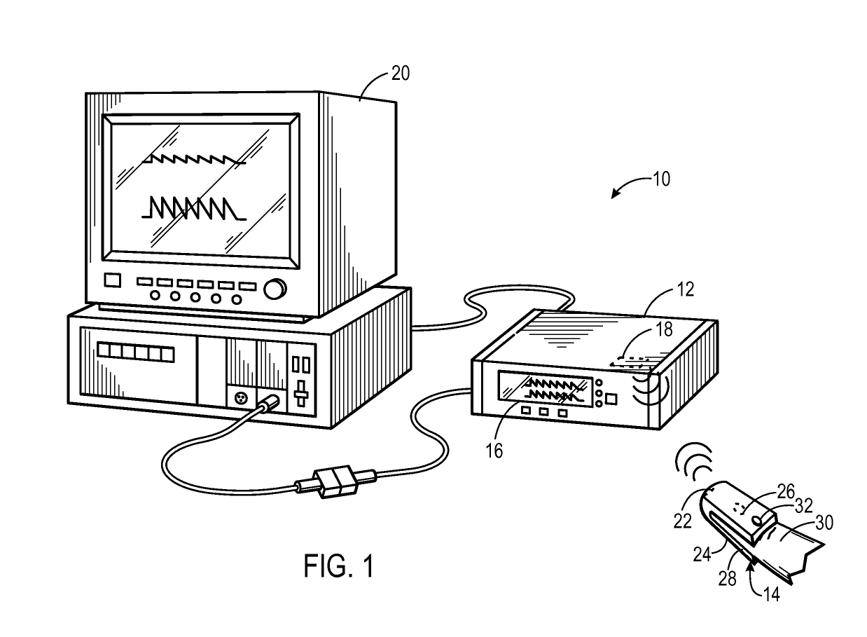

One embodiment of a patient monitoring system 10 that may benefit from the approaches described herein is depicted in FIG. 1. The illustrated patient monitoring system 10 includes a patient monitor 12 and a wireless patient sensor 14. The patient monitoring system 10 is configured to enable the calculation of one or more physiological parameters of a patient by either of the wireless patient sensor 14 or the patient monitor 12. For example, in one embodiment, the patient monitoring system 10 may be configured to enable the wireless patient sensor 14 to perform various calculations in order to limit wireless communication and to conserve battery power in the wireless patient sensor 14. Although the illustrated embodiment of system 10 is a pulse oximetry monitoring system, it should be noted that the patent monitoring system 10 may be configured to perform any number of measurements on a patient to determine one or more physiological parameters of the patient. That is, while the pulse oximetry monitoring system 10 may determine pulse rates and blood oxygen saturation levels (e.g., SpO₂ values) for a patient, the system 10 may, additionally or alternatively, be configured to determine a patient's respiration rate, glucose levels, hemoglobin levels, hematocrit levels, tissue hydration, patient temperature, cardiogram information, blood pressure, or pulse transit time, as well as other physiological parameters. Furthermore, while the illustrated embodiment includes the wireless patient sensor 14 that communicates with the patient monitor 12 in a wireless fashion, the present approaches are also applicable to configurations in which a patient sensor communicates with the patient monitor 12 using a wired connection. Such communication may be carried out using light guides, one or more conductors (e.g., via a cable), or any other suitable communication and/or power transmission features. In such embodiments, either or both of the patient monitor 12 and wireless patient sensor 14 may perform any of the determinations or calculations described herein.

The patient monitor 12 may include a display 16, a wireless module 18 for transmitting and receiving wireless data, a memory, a processor, and various monitoring and control features. Based on data received from the wireless patient sensor 14, the patient monitor 12 may display physiological parameters of the patient on display 16. The system 10 may also be communicatively coupled to a multi-parameter monitor 20 to facilitate presentation of patient data, such as pulse oximetry data determined by system 10 and/or physiological parameters determined by other patient monitoring systems (e.g., electrocardiographic (ECG) monitoring system, a respiration monitoring system, a blood pressure monitoring system, etc.). For example, the multi-parameter monitor 20 may display a graph of SpO₂ values, a current pulse rate, a graph of blood pressure readings, an electrocardiograph, and/or other related patient data in a centralized location for quick reference by a medical professional.

In certain embodiments, the wireless patient sensor 14 may be completely or partially disposable. That is, in certain embodiments, a portion of the wireless patient sensor 14 may be disposed after patient use. In certain embodiments, the wireless patient sensor 14 may be constructed in a modular fashion such that portions of the sensor 14 (e.g., processing circuitry) may be removed to be recycled into other sensors while other portions (e.g., the body) of the sensor 14 are disposed. In some embodiments, the sensor 14 may include one or more features that contact and/or pierce the patient's skin. For example, the sensor 14 may include microneedles and/or light guides configured to penetrate and, in certain configurations, traverse a skin layer (e.g., the stratum corneum) into underlying tissue. Accordingly, those features may be at least a portion of what may be discarded after use.

Like the patient monitor 12, the patient sensor 14 also includes a wireless module 22. The wireless module 22 of the sensor 14 may establish wireless communication with the wireless module 18 of the patient monitor 12 using any suitable protocol. For example, the wireless modules 18, 22 may be capable of communicating using the IEEE 802.15.4 standard, and may be, for example, ZigBee, WirelessHART, or MiWi modules. Additionally or alternatively, the wireless modules 18, 22 may be capable of communicating using the Bluetooth standard, one or more of the IEEE 802.11 standards, an ultra-wideband (UWB) standard, or a near-field communication (NFC) standard. In certain embodiments, the wireless module 22 of the patient sensor 14 may be used to transmit either raw detector signals or calculated physiological parameter values to the patient monitor 12 depending on the noise level and/or complexity of the detector signal.

The illustrated wireless patient sensor 14 includes an emitter 24 and a detector 26 coupled to a body 28 of the sensor 14. The body 28 of the wireless patient sensor 14 facilitates attachment to a patient tissue (e.g., a patient's finger, ear, forehead, or toe). For example, in the illustrated embodiment, the sensor 14 is configured to attach to a finger of a patient 30. When attached to a pulsatile tissue such as the finger, the emitter 24 may transmit light at certain wavelengths (e.g., for example, RED light and/or IR light) into the tissue, wherein the RED light may have a wavelength of about 600 to 700 nm, and the IR light may have a wavelength of about 800 to 1000 nm. The detector 26 may receive the RED and IR light after it has passed through or is reflected by the tissue. The emitter 24 may emit the light using one, two, or more LEDs, or other suitable light sources. The detector 26 may be any suitable light detecting feature, such as a photodiode or photo-detector. The process of emission and detection of the light after passing through or reflection by the tissue is used to characterize the nature of the underlying tissue, as the amount of light that passes through the patient tissue and other characteristics of the light may vary according to the changing amount of certain blood constituents in the tissue. In addition to the emission and detection features noted above, the wireless patient sensor 14 may include a button or switch 32 which may be used to activate (e.g., turn on the emitter 24 and detector 26) and deactivate the sensor 14 (e.g., turn off the emitter 24 and detector 26) to conserve battery power.

It should be noted that the amount of power used by the sensor 14 during the process of transmission and/or reflection of the IR and/or RED light may depend at least partially on the efficiency with which the light is able to pass through the tissue. That is, because pulse oximetry relies on monitoring the oxygenated blood underlying the patient's skin, the light typically first passes through the skin, into the underlying perfuse (i.e., blood-filled) tissue, and back out through the skin. Therefore, a portion of the light emitted by the emitter 24 may be attenuated or otherwise absorbed by the skin, which increases the minimum level of power suitable for driving the emitter 24 while also providing a suitable signal-to-noise ratio. Accordingly, as discussed in detail below, the emitter 24 may include or may be in communication with optical elements that reduce the attenuation of the emitted light by the patient's skin. These optical elements may result in an improvement in the sensor efficiency by reducing the power drawn by the sensor 14.

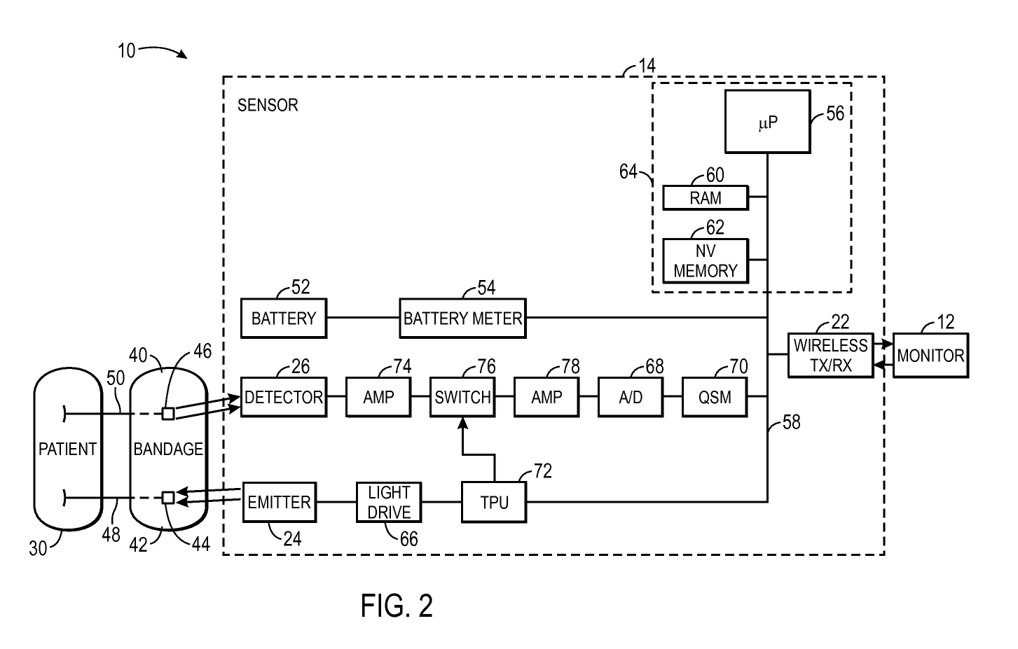

In accordance with one example, FIG. 2 illustrates a block diagram of plurality of components that may be present within the body 28 of the wireless patient sensor 14 to facilitate the acquisition, processing, and transmission of physiological data (e.g., a plethysmographic signal). In addition, as illustrated, the wireless patient sensor 14 may work in conjunction with a bandage 40 having one or more biodegradable light guide elements configured to transmit light between the wireless patient sensor 14 and the patient 30. In particular, the bandage 40 of FIG. 2 includes a body 42, an emitter lens portion 44 (e.g., an emitter opening), a detector lens portion 46 (e.g., a detector opening), and first and second light guides 48, 50 coupled to or inserted within the emitter lens portion 44 and the detector lens portion 46, respectively. As discussed in detail below with respect to FIG. 4, any one or a combination of these elements may be formed from one or more biodegradable materials.

While illustrated as separate from the patient 30, in accordance with present embodiments, the bandage 40 may attach (e.g., via an adhesive) to the patient 30 so as to prevent light shunting between the emitter and detector lens portions 44, 46 and also to mitigate optical interference from ambient light. In addition, the bandage 40 may be in physical contact with the wireless patient sensor 14 to facilitate light transmission therebetween. For example, the bandage 40 and the wireless patient sensor 14 may be coupled via an adhesive or hook-and-loop connector, or may simply be placed against one another. Regardless of the method used to place the wireless patient sensor 14 and the bandage 40 into physical contact, in one embodiment, they are in contact in a manner that mitigates ambient light interference and light shunting between the emitter 24 and the detector 26.

During operation, the emitter 24 emits one or more wavelengths of light used for pulse oximetry (e.g., IR and/or RED light), which is received at the emitter lens portion 44 of the bandage 40. The emitted light is then transmitted along the first light guide 48 and into the patient 30. The light then travels through the underlying perfuse tissue, where at least a portion of the light is absorbed. The second light guide 50 then transmits the resulting light to the detector lens portion 46, where the light is then communicated to the detector 26. The arrangement of the wireless patient sensor 14 and the bandage 40 is not limited to the particular illustrated embodiment. For example, the wireless patient sensor 14 may be used for regional oximetry, wherein the wireless patient sensor 14 may include two or more detectors for monitoring the oxygen perfusion in a region of the patient's tissue. In such embodiments, the bandage 40 may have one, two, three, or more detector lens portions each matched to one or more detectors and one or more light guides of the wireless patient sensor 14. Furthermore, embodiments in which the wireless patient sensor 14 includes more than one emitter are also presently contemplated. In such embodiments, the bandage 40 may have one, two, three, or more emitter lens portions each matched to one or more emitters and one or more light guides of the wireless patient sensor 14.

In addition to the wireless patient sensor 14 and the bandage 40, the system 10 includes the patient monitor 12. The wireless patient sensor 14 may be communicatively connected to the patient monitor 12 via wireless communication, as discussed above. Again, the wireless patient sensor 14, the patient monitor 12, or a combination thereof, may process the measurements performed by the wireless patient sensor 14 to determine patient physiological data. A battery 52 may supply the wireless patient sensor 14 with operating power for the emission, detection, communication, and any processing performed by the wireless patient sensor 14. By way of example, the battery 52 may be a rechargeable battery (e.g., a lithium ion, lithium polymer, nickel-metal hydride, or nickel-cadmium battery) or may be a single-use battery such as an alkaline or lithium battery. Since the techniques described herein may enable reduced battery consumption, the battery 52 may be of a lower capacity, and accordingly much smaller and/or cheaper, than a battery needed to power a similar wireless sensor that does not employ the disclosed embodiments. A battery meter 54 may provide the expected remaining power of the battery 52 to a processing device of the wireless patient sensor 14 and/or the monitor 12.

In the illustrated embodiment, the wireless patient sensor 14 also includes a microprocessor 56 coupled to a main system bus 58 that controls the operation of the sensor 14. In general, the processor 56 may be a low-power processor compared to the processor that may be present within the patient monitor 12. By way of example, the processor 56 may be an 8-bit or 16-bit micro-controller or an 8-bit or 16-bit processor, while the processor of the patient monitor 12 may be a 32-bit or 64-bit processor, such as those used in monitors available from Nellcor.

The processor 56 may work in conjunction with random access memory (RAM) 60 and/or non-volatile (NV) memory 62, which also may be connected to the system bus 58. The RAM 60 may be implemented using low-power memory modules, and may be 8-bit or 16-bit addressable for use with the processor 56. In certain embodiments, the RAM 60 may be implemented as a memory that is part of the processor 56, or RAM 60 may be a designated portion of NV memory 62. NV memory 62 may be an electrically erasable programmable read-only memory (EEPROM) or flash memory storage device. In the illustrated embodiment, the processor 56, RAM 60, and NV memory 62 are incorporated into processing circuitry 64. That is, in certain implementations, the processor 56, RAM 60, and/or NV memory 62 may be included within a single chip within the sensor 14. Indeed, in certain embodiments, the processing circuitry 64 may also include the system bus 58, a time processing unit (TPU) 72, an A/D converter 68, a queued serial module (QSM) 70, and/or the wireless module 22 within a single chip. These components are discussed in further detail below.

In an embodiment, the NV memory 62 may include one or more sets of instructions to be executed by the processor 56 for determining patient physiological parameters based on the data obtained from the emission and subsequent detection of light by the optical elements of the wireless patient sensor 14. That is, based at least in part on the signals provided by the detector 26, the microprocessor 56 may calculate a physiological parameter of interest using various algorithms and coefficient values that may be stored in NV memory 62. Additionally, NV memory 62 and/or RAM 60 may also store historical data and/or values (e.g., detector signal data, data points, trend information) for the physiological parameter of the patient. For example, the NV memory 62 and/or RAM 60 may store calculated SpO₂ values (e.g., one value per minute) for the most recent twenty minutes of sensor operation. The processor 56 may use these stored values to determine the variance in a patient physiological parameter (e.g., SpO₂). By further example, NV memory 62 and/or RAM 60 may be used to temporarily store or buffer the detector signal data and/or calculated physiological parameter values for a period of time, such as if the wireless connection between the wireless patient sensor 14 and the patient monitor 12 is interrupted. Accordingly, upon reestablishing wireless communications, the sensor 14 may send the buffered data to the monitor 12 in a quick burst before resuming normal operations. A similar buffer mechanism may also be employed, for example, if the processor 56 is temporarily lagging behind on detector signal data processing. In such circumstances, when the processor 56 becomes available, or when the patient sensor 14 transfers the data to the monitor 12 for processing, the detector signal data may be appropriately processed without resulting in a gap in patient's physiological data.

The algorithms stored in NV memory 62 may be used to determine the physiological parameter of the patient using the low-power processor 56 of the wireless patient sensor 14. These algorithms may include those disclosed in U.S. Pat. No. 4,911,167, filed Mar. 30, 1988, U.S. Pat. No. 6,411,833, filed Nov. 5, 1999, which are incorporated by reference herein in their entirety. For example, in the case of a pulse oximetry patient monitoring system 10, the NV memory 62 may include algorithms that calculate a SpO₂ value using a ratio-of-ratios calculation, in which the SpO₂ value is equal to the ratio of the time-variant (AC) and the time-invariant (DC) components of the detector signal acquired using RED light divided by the ratio of the AC and DC components of the detector signal acquired using IR light.

In general, a number of processing algorithms may be used to determine the AC and DC components of the detector signal. For example, the DC components of the detector signals may be determined using a number of different methods, including a moving average over a defined time window, an infinite impulse response (IIR) Butterworth low-pass filter, or using a minimum plethysmograph value over a defined time window. Furthermore, for such a calculation, the AC component may be determined using a number of different methods, such as using an average of local plethysmograph derivatives over a period of time, using a derivative-base peak identification and subsequently determining the difference between the amplitude and nadir of each pulse, using a difference in the maximum and minimum values of the plethysmograph waveform over a period of time, and/or using a fast Fourier transform (EFT) with subsequent amplitude analysis. It should be noted that the aforementioned processing algorithms are provided as examples, and patient monitoring system 10 may utilize any number of algorithms as would be known to one of ordinary skill in the art.

Additionally, the NV memory 62 may store caregiver preferences, patient information, and various operational parameters of the wireless patient sensor 14. For example, the NV memory 62 may store information regarding the wavelength of one or more light sources of the emitter 24, which may allow for selection of appropriate calibration coefficients for calculating a physiological parameter (e.g., blood oxygen saturation). Furthermore, in an embodiment, these calibration coefficients and/or calibration curves may also be stored in the NV memory 62 after they have been determined through empirical calibration of the sensor 14 during or after manufacturing. Additionally, in circumstances where the monitor 12 will be calculating the physiological parameter of the patient 30, NV memory 62 may also provide information regarding the emitter wavelengths, calibration coefficients, and/or calibration curves to the patient monitor 12 via the wireless connection between the sensor 14 and monitor 12.

During operation of the wireless patient sensor 14, the TPU 72 may provide timing control signals to light drive circuitry 66 to control when the emitter 24 is illuminated, and if multiple light sources are used, the multiplexed timing for the different light sources. The TPU 72 may also control the gating-in of signals from the detector 26 through an amplifier 74 and a switch 76. In embodiments where multiple light sources are used, the switch 76 may ensure that signals are sampled at the proper time, depending upon which of the multiple light sources is illuminated. After passing through the switch 76 and a subsequent amplifier 78, the signal may pass through the analog-to-digital converter 68 before arriving at the QSM 70. The digitized detector signal may be collected and temporarily stored in the QSM 70 for later downloading into RAM 60 as the QSM 70 fills up. In an alternative embodiment, the processor 56 may receive the digitized detector signal directly from the A/D converter 68, without the use of the QSM 70, and transfer it to RAM 60 for processing. The processor 56 may also use a built-in Direct Memory Access (DMA) peripheral to perform the data transfer in the background while the core is in a low-power or sleep mode, or while the core is processing previously received A/D samples.

As noted above, either the wireless patient sensor 14 or the patient monitor 12 may implement any one or a combination of the above processes. Indeed, in certain embodiments, such as when a wired sensor is coupled to the patient monitor 12, the monitor 12 may include a number of the components described above, or similar components that perform substantially the same functions to those described above. For example, the monitor 12 may include features identical to or otherwise having similar functionality to the processing circuitry 64, the light drive circuitry 66, TPU 72, amplifier 74, switch 76, amplifier 78, analog-to-digital converter 68, QSM 70, or any combination thereof.

Regardless of the particular configuration employed, in RAM 60, the digitized detector signal may be divided into portions and stored or encoded in such a way to include details regarding the detector signal and/or the detector signal acquisition. In certain embodiments, the digitized detector signal may be divided into portions and stored in RAM 60 based on the wavelength of light emitted when acquiring the detector signal. For example, a portion of a digitized detector signal may be stored in RAM 60 along with data indicating that the signal was acquired when the emitter 24 was emitting a particular wavelength of light (e.g., RED or IR) into the tissue of the patient 30. Alternatively, in certain embodiments, the digitized detector signal may include a continuous stream of detector signals acquired using two or more wavelengths of light (e.g., RED and IR). In such embodiments, a set of timing data, representing the activities of the TPU 72, light drive 66, and/or emitter 24 during detector signal acquisition, may be stored in RAM 60 so that a processor (e.g., processor 56) may use this timing data to deconvolute the digitized detector signal into the component detector signals for each wavelength emitted. Accordingly, when the detector signal data is subsequently processed by the processor 56 of the wireless patient sensor 14, or by the processor of the patient monitor 12, the included signal acquisition details may ensure that appropriate portions of the digitized detector signal are used in the appropriate point in the calculation when determining the physiological parameter of the patient 30. As such, for clarity, the term “detector signal data” is used herein to describe the digitized detector signal or filtered detector signal combined with any other signal acquisition details that may be used to interpret the digital detector signal. For example, the detector signal data may, in addition to a digitized detector signal, incorporate emitter wavelength information, timing data, calibration coefficients, or calibration curves that may be used by a processor (e.g., processor 56) to process the digitized detector signal and/or calculate the physiological parameter of the patient 30.

Once the detector signal data has been stored in RAM 60, it may be further processed by the processor 56 of the wireless patient sensor 14 to determine specific patient physiological parameters of interest, such as pulse rate, blood oxygen saturation, and so forth. In addition, because the first and second light guides 48, 50 are biodegradable and thus, will degrade over time, the processor 56 may also determine, based on the detector signal data, whether the whole bandage 40, or components of the bandage 40, might need to be changed to ensure accurate measurements. By way of non-limiting example, the processor 56 may recognize wide variations in the data over relatively short amounts of time, a steep drop in signal-to-noise ratio, or an indication that the data is contaminated with noise (e.g., from ambient light). Any one or a combination of these and other factors might indicate that the bandage 40 is no longer suitable for use. In such situations, the wireless module 22 may communicate a signal to the patient monitor 12 to provide a user-perceivable indication (e.g., a visual or audible indication) to inspect and, where appropriate, change the bandage 40. By way of example, the patient monitor 12 may display a message on the display 16 (FIG. 1) to inspect and/or replace the sensor 14 and/or bandage 40. In certain embodiments, such as where the wireless patient sensor 14 incorporates elements of the bandage 40 (e.g., light guides that contact or otherwise pierce the skin), the user-perceivable indication by the monitor 12 may indicate that the sensor 14 should be changed. Additionally or alternatively, the sensor 14 may provide the indication as a blinking light, or a light having a different color than a light indicating normal operation of the sensor 14.

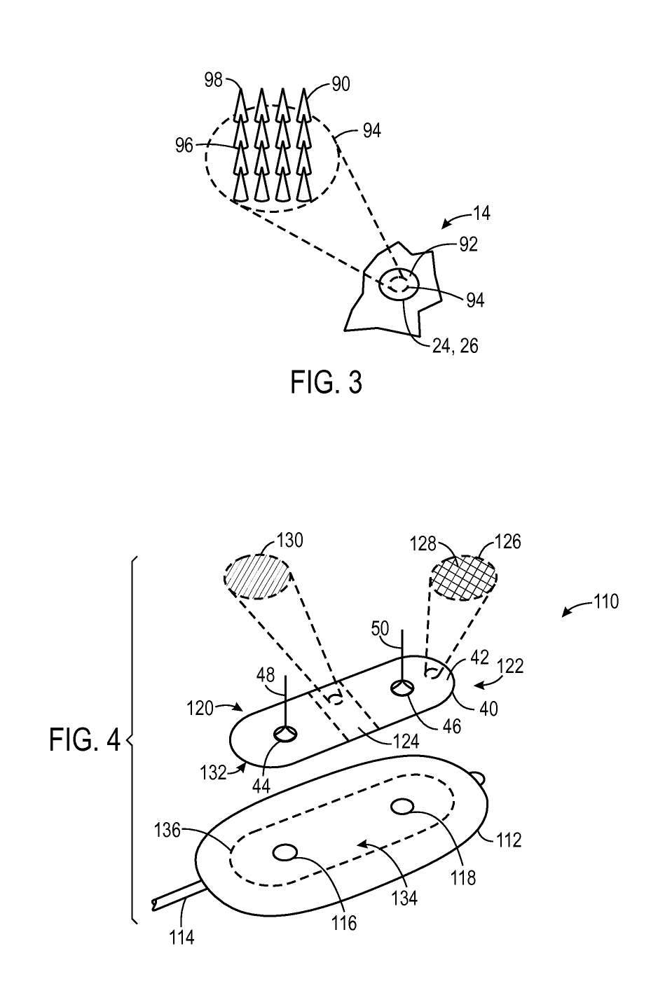

As noted above, the wireless patient sensor 14, or any such sensor used according to the present disclosure, is not limited to being used in conjunction with the bandage 40 depicted in FIG. 2. Rather, other embodiments may be used in addition to, or in lieu of the bandage 40 to provide enhanced light transmission through the patient 30. One such embodiment is depicted in FIG. 3, which illustrates an embodiment of a portion of the emitter 24 or the detector 26 of the wireless patient sensor 14 as having microneedles 90 formed from a biodegradable material, such as poly(lactic acids), synthetic and natural silks, cellulose and its derivatives, chitin and chitosan derivatives, alginates, sugars, and poly(hydroxyalkanoates), or any combination thereof. In one embodiment, the biodegradable material is silkworm silk or, more specifically, fibroin isolated from silkworm silk. In a general sense, the material used to produce the microneedles 90 will enable the microneedles 90 to have an index of refraction that is greater than the index of refraction of the patient's blood or tissue. Such an index of refraction may be desirable to enable the microneedles 90 to act as a light guide through the tissue surrounding the microneedles 90 after penetration.

In the illustrated embodiment, the microneedles 90 are positioned on a lens 92 of the emitter 24 or detector 26, and may be provided as a layer 94 that is adhered to the lens 92, or may be integrated into the construction of the lens 92. For example, in situations where the optical portions of the sensor 14 are reusable, it may be desirable to adhere the layer 94 of the microneedles 90 to the outer surface of the lens 92 to enable the layer 94 to be removed for cleaning and reuse of the sensor 14, and subsequent replacement of an unused set of microneedles 90. However, in embodiments in which the sensor 14 is disposable, the microneedles 90 and the lens 92 may both be constructed from a biodegradable material.

Microneedles 90 positioned on the emitter 24 may be light guides that aid in the transmission of light of predetermined wavelengths from the emitter 24 and into the patient 30, while microneedles 90 positioned on the detector 26 may also act as light guides that transmit light from the perfuse tissue to the detector 26. In particular, the microneedles 90, as illustrated, may have a cross-sectional geometry in which each microneedle 90 includes a base 96, which are each configured to be disposed proximate the emitter 24 or detector 26, and a tip 98 for penetrating the patient's skin to a desired depth. The illustrated triangular cross-sectional geometry may arise from the microneedles 90 having any tapered shape, including wedge shapes, cone shapes, pyramid shapes, or any combination. The shapes may alternatively be cylindrical or any other suitable shape, such as a combination of cylindrical and tapered shapes, or any shape or combination of shapes facilitating penetration into the patient's tissue (e.g., skin).

The length of each of the microneedles 90, defined by the distance from the widest portion of the base 96 to the thinnest portion of the tip 98, may be any suitable length for providing a desired amount of penetration into the patient's skin, such as to penetrate an outer layer of the patient's skin (e.g., the stratum corneum) to mitigate absorbance by the skin and enhance transmission into the underlying perfuse tissue. In another embodiment, the microneedles 90 may be suitably sized for use in neonatal care. In such embodiments, in some embodiments, the microneedles 90 may be used to penetrate the neonate's skin or semi-soft tissue, such as the neonate's skull (e.g., relatively unformed portions of the skull). By way of non-limiting example, the length of the microneedles 90 may be between approximately 500 nm and 2000 μm, such as between approximately 1 μm and 1500 μm, or between 200 μm and 1000 μm. In a general sense, shorter distances may be chosen for smaller patients (e.g., neonates) or patients with sensitive skin, which results in a smaller degree of penetration, while longer distances may be chosen for adult sensors for a greater degree of penetration.

The width of the tip 98 of each of the microneedles 90 may be any suitable size so as to enable penetration of the skin while also enabling a desired degree of robustness to enable monitoring for a desired amount of time. By way of non-limiting example, the tip 98 may have a width of between approximately 10 nm and 50 μm, such as between approximately 50 nm and 10 μm, or between approximately 100 nm and 1 μm. In addition, the width of the microneedles 90 may vary along the length of the microneedles 90, or may be the same.

The microneedles 90 may be formed using any process suitable for producing the microneedles 90 having the desired materials at the desired geometries. Such processes may include, but are not limited to, laser-initiated polymerization (e.g., localized polymerization) or micromolding. In laser-initiated polymerization, the microneedles 90 may be formed using a bulk suspension or solution having a suitable monomeric mixture including biodegradable materials. The suspension or solution may then be subjected to localized polymerization using one or more lasers that scan the suspension or solution to create the microneedles 90 by initiating polymerization at specific locations. In other embodiments, such as where the microneedles 90 are formed using molding techniques, a micromold having a substrate and indentations defining the geometry of the microneedles 90 may be used. A bulk suspension or solution having the desired materials (e.g., an aqueous solution having fibroin isolated from silk) may be poured or otherwise disposed in the micromold, and subsequently dried. After drying, the resulting structure having the microneedles may be removed from the mold.

The processes used to produce the microneedles 90 may also be varied to incorporate other desired materials into the microneedles 90. For example, the rate at which the microneedles 90 degrade may be at least partially controlled using one or more additional materials in the bulk suspension or solution used for the polymerization and/or molding. For example, in one embodiment, a biodegradable polymer having a known rate of degradation may be blended into the bulk suspension or solution in a known amount to affect the rate at which the microneedles 90 degrade. Additionally or alternatively, one or more pharmaceutical or “active” agents may be incorporated into the bulk suspension or solution used to produce the microneedles 90. The pharmaceutical agents may be used to facilitate healing of the tissue being monitored (e.g., a post-operative internal tissue), for pain relief, clotting, or any other use.

As noted above, the microneedles 90 facilitate transmission of the light emitted by the emitter 24 into the patient's perfuse tissue, and from the tissue to the detector 26 in a manner that mitigates absorbance by the outer layers of the patient's skin. Further, the microneedles 90 are attached directly to, or are integral with a lens of the emitter 24 and/or the detector 26. However, in some embodiments, it may be desirable to provide a bandage that may be kept attached to the patient 30 separately from the sensor 14. For example, it may be desirable to change the sensor 14 without also having to re-insert light guides, etc. into the patient to change sensors (e.g., due to sensor malfunctions, low sensor batteries, or another sensor condition). Accordingly, as discussed with respect to FIG. 2 above, the present disclosure also provides embodiments of a bandage having one or more light guides that are insertable into a patient tissue.

One embodiment of the bandage 40 is depicted in FIG. 4. In particular, FIG. 4 depicts a sensor assembly 110 in which the bandage 40 is usable in conjunction with a patient sensor 112 configured to communicate with the patient monitor 12 via a sensor cable 114, although the sensor 112 may have any suitable communication features (e.g., wireless communication modules). The illustrated bandage 40 includes the emitter lens portion 44, the detector lens portion 46, and the first and second light guides 48, 50. In a similar manner to the configuration discussed above with respect to FIG. 2, the emitter lens portion 44 has a position corresponding to an emitter 116 of the wired patient sensor 112 and the detector lens portion 46 has a position corresponding to a detector 118 of the sensor 112. The relative positioning between the bandage 40 and the wired patient sensor 112 enables the bandage 40 to transmit light from the emitter 116 through the first light guide 48, and also enables the bandage 40 to receive light through the second light guide 50 to the detector 118.

As noted above, any one or a combination of the optical elements of the bandage 40, including the emitter lens portion 44, the detector lens portion 46, and the first and second light guides 48, 50, may include a biodegradable material. By way of non-limiting example, these materials may include poly(lactic acids), synthetic and natural silks, cellulose and its derivatives, chitin and chitosan derivatives, alginates, sugars, and poly(hydroxyalkanoates), or any combination thereof. In one embodiment, the optical elements may be produced using solutions containing fibroin, which may be isolated from naturally produced silkworm silk. Generally, the optical elements will be biodegradable, and may not induce an immunological response when implanted in the patient's body. Further, such a material may be useful in constructing the first and second light guides 48, 50, at least because fibers produced using fibroin may have an index of refraction that is greater than the index of refraction of blood, meaning that the first and second light guides 48, 50 may transmit light along their length without substantial losses (e.g., 20% or less, or 10% or less losses) to the surrounding environment (e.g., the patient's blood or internal tissues).

The first and second light guides 48, 50 may have any dimension, such as any suitable diameter, cross-sectional thickness, length, or the like, suitable for placement within the patient, attachment to internal patient tissues, and protrusion through the skin. By way of non-limiting example, the diameter or cross-sectional thickness of each of the first and second light guides 48, 50 may be any dimension suitable for transmission of light between the patient's tissue and the sensor 14 while also being minimally invasive for the patient. Such dimensions may be between approximately 50 nm and 2000 μm, between approximately 100 nm and 1000 μm, or between approximately 1 μm and 500 μm. Similarly, the length of the first and second light guides 48, 50 may be any suitable length for optically coupling a tissue of interest (e.g., an internal tissue) with the bandage 40, such as between approximately 100 μm and 30 cm, or between approximately 1 mm and 15 cm.

The features of the bandage 40 used to facilitate light transmission from the emitter 116 to the detector 118 through the patient's tissue are positioned within (e.g., are integral with) or on the body 42 of the bandage 40. The body 42 may include one or more biodegradable materials, or may include conventional materials such as polymer materials suitable for use in medical contexts. For example, in the illustrated embodiment, the body 42 includes an emitter region 120 and a detector region 122 separated by a light barrier 124. The emitter and detector regions 120, 122 of the bandage 40 may be formed using one or more biodegradable materials (e.g., fibroin isolated from silkworm silk), as depicted by the expanded region 126. Further, these regions may be woven in a manner that enables the patient's skin to breathe (e.g., be exposed to the air) through the bandage 40, which enables the bandage 40 to be worn for an extended period of time.

The emitter and detector regions 120, 122 may be formed using the same or different materials, and may independently be transparent to the light emitted by the emitter 116, or may be opaque to the light emitted by the emitter 116. By way of non-limiting example, it may be desirable for the emitter and detector regions 120, 122 to be transparent to the light emitted by the emitter 116 to enable greater dispersion of the light into the patient's tissue at the emitter region 120, and also to enable a wider area for collecting light that has passed through the patient's tissue at the detector region 122. In other embodiments, it may be desirable for the emitter and detector regions 120, 122 to be opaque to the light emitted by the emitter 116 to enable the light to be more directly transmitted into a particular tissue of interest rather than the entirety of the tissue underlying the bandage 40. Similarly, it may be desirable for the detector region 122 to be opaque to the transmitted light to enable only light conducted along the light guide 50 to pass to the detector 118 (e.g., from the tissue of interest). Further, opacity of the emitter and detector regions 120, 122 may be desirable to prevent contamination from ambient light.

The particular arrangement of the opacity or transparency of either or both of the emitter region 120 and the detector region 122 may be chosen based on these or other factors. By way of further example, the materials used to construct the emitter and detector regions 120, 122 to enable the properties described herein may independently include biodegradable materials such as any one or a combination of poly(lactic acids), synthetic and natural silks, cellulose and its derivatives, chitin and chitosan derivatives, alginates, sugars, and poly(hydroxyalkanoates), or any combination thereof, or conventional polymeric materials such as polyolefins, polyesters, polyvinylhalides, polyurethanes, polyamides, or the like. One example of a biodegradable optically conductive region is a region formed by weaving fibroin into a structure capable of acting as a support layer for the bandage. Conversely, one example of a breathable, biodegradable optically transparent region is a region formed using a biodegradable polymer that is opaque to the ambient and emitted light, and may be expanded so as to form internal pores having a size suitable for the transmission of only water vapor and other such gases (e.g., oxygen, nitrogen) to enable some degree of breathability. The opaque region might include distinct layers of such porous biodegradable polymers arranged in an alternating fashion (e.g., such that the pores do not align) so as to prevent light from traversing the pores. Additionally or alternatively, a foam structure may be internally positioned within the particular region to afford some degree of cushioning, breathability, and opacity. The foam structure may also be formed using any one or a combination of the materials noted above.

In embodiments where the emitter and detector regions 120, 122 are formed from the same materials and are opaque to ambient light and the light emitted by the emitter 116, the emitter region 120, the detector region 122, and the light barrier 124 may be one continuous structure. Similarly, in embodiments where one of the emitter and detector regions 120, 122 is opaque to the ambient and emitted light, the opaque emitter and/or detector regions 120, 122 may be a continuous structure with the light barrier 124. However, in embodiments where there is a possibility of light shunting between the emitter 116 and the detector 118, the light barrier 124 may be formed from a different material, may be woven differently, or a combination thereof, compared to the emitter and detector regions 120, 122, as depicted in expanded region 130.

Biodegradable or non-biodegradable materials may also be used for coupling the bandage 40 to the sensor 112. For example, in embodiments where the bandage 40 and the sensor 112 couple to one another, an adhesive, a hook-and-loop attachment mechanism, or other attachment mechanism, may be used to secure the bandage 40 to the sensor 112. In particular, in an embodiment, a biodegradable or non-biodegradable adhesive may be provided on a non-patient contacting surface 132 of the bandage 40 and/or on a bandage-contacting surface 134 of the sensor 112. It should be appreciated that the bandage-contacting surface 134 of the sensor 112 may be a surface that would otherwise contact the patient, and may be suitably modified for attachment to the bandage 40. In another embodiment, the sensor 112 may be an un-modified conventional sensor, and the non-patient contacting surface 132 of the bandage may be responsible for the coupling between the bandage 40 and the sensor 112. In embodiments where the bandage 40 and the sensor 112 couple by a hook-and-loop mechanism, the hook or the loop may be positioned on the non-patient contacting surface 132 of the bandage 40. For example, in one embodiment, at least a portion of the body 42 of the bandage 40 may be formed using one or more woven materials that facilitate attachment by acting as a loop portion of a hook-and-loop attachment mechanism. The corresponding hook portion may be secured to the sensor 112 in a region 136 having a geometry generally corresponding to the bandage 40. Conversely, the non-patient contacting surface 132 of the bandage 40 may include a hook portion, while the region 136 of the sensor 112 corresponding to the bandage 40 may include the loop portion of a hook-and-loop mechanism.

It should be noted that the density of the hook and loop mechanism used to couple the bandage 40 and the sensor 112 may, in certain embodiments, be selected so as to mitigate inadvertent removal of the bandage 40 from the patient 30. For example, in one embodiment, the density of the hook and loop mechanism may be chosen such that the coupling strength between the bandage 40 and the sensor 112 is less than, such as between 10% and 95% of, the bonding strength of the mechanism used to couple the bandage 40 to the patient 30 (e.g., an adhesive strength). In embodiments where the density of the hook and loop mechanism is chosen such that the coupling strength between the bandage 40 and the sensor 112 is greater than the bonding strength of the mechanism used to couple the bandage 40 to the patient 30, it should be appreciated that the sensor 112 may be removed from the bandage 40 while maintaining the bandage 40 against the patient 30 by pressing the bandage 40 against the patient 30 while removing the sensor 112 from the bandage 40. In still further embodiments, it may be desirable to remove both the sensor 112 and the bandage 40 from the patient 30. In such embodiments, the strength of the coupling mechanism (e.g., the hook-and-loop mechanism) used to couple the bandage 40 and the sensor 112 may be less than, equal to, or greater than, the strength of the coupling mechanism used to secure the bandage 40 to the patient 30.

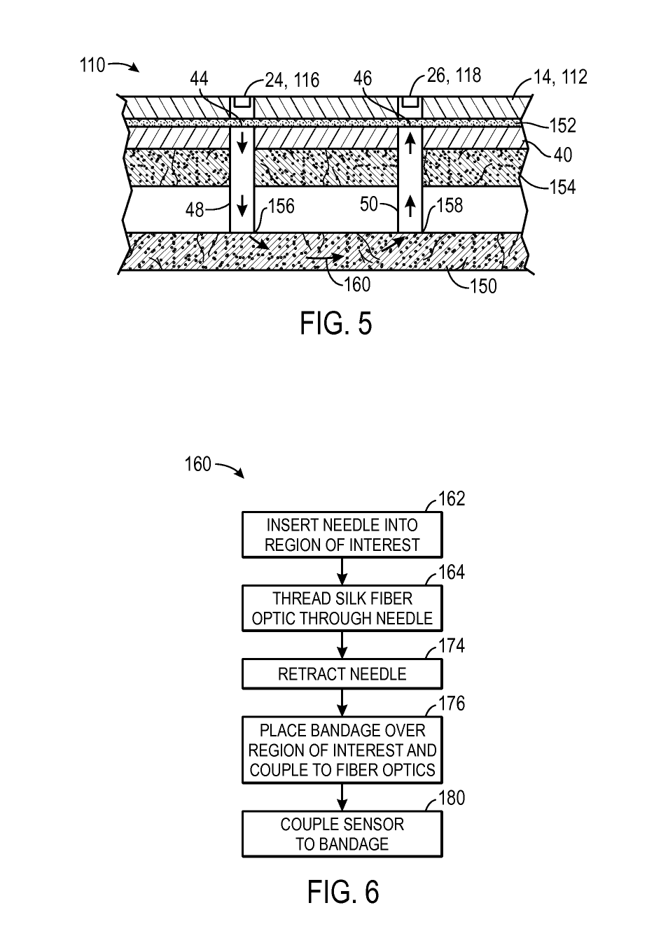

Regardless of the manner in which the bandage 40 and the sensor 112 are secured or otherwise in physical contact with one another, the sensor assembly 110 formed from their combination may be used to monitor various parameters (e.g., SpO₂) of an internal tissue 150, as depicted in the cross-sectional view of FIG. 5. In particular, FIG. 5 depicts the relative arrangement of the components of the sensor assembly 110 in relation to the patient 30 during monitoring. However, it should be noted that the illustrated arrangement is merely one aspect, and that other arrangements are also presently contemplated, as discussed in detail below. Moving from the top to the bottom of FIG. 5, the depicted arrangement includes the sensor assembly 110 having the wireless patient sensor 14 or the wired patient sensor 112 coupled to the bandage 40 via a bonding layer 152. The bonding layer 152 may generally correspond to the features discussed above that may be disposed on the non-patient contacting surface 132 of the bandage 40 and/or the bandage contacting surface 134 of the sensor 112. Thus, the bonding layer 152 may be a biodegradable or non-biodegradable adhesive layer, a hook-and-loop attachment mechanism, or any other suitable mechanism for coupling the bandage 40 to the sensor 14, 112.

The bandage 40 of the sensor assembly 110 is illustrated as being positioned directly in contact with a skin layer 154 of the patient 30, though it should be noted that the bandage 40 may be removably secured to the patient 30 using any suitable adhesion mechanism, including a hypoallergenic or otherwise biocompatible and, in some embodiments, biodegradable adhesive layer. The emitter and detector lens portions 44, 46, as noted above, are positioned so as to enable light to enter into the first and second light guides 48, 50. As illustrated, the first and second light guides 48, 50 may traverse the skin layer 154 (e.g., via corresponding openings in the outer skin layer 154 created by a needle). The skin layer 154 may include one or more layers of the epidermis (e.g., the stratum corneum, stratum germinativum), the dermis, and, in certain embodiments, the hypodermis (subcutis). The first and second light guides 48, 50 are also depicted as being attached to (or otherwise in contact with) the internal tissue 150 at their respective ends 156, 158. In particular, the first and second light guides 48, 50 are depicted as being implanted in the patient so as to enable transmission of light emitted by the emitter 24, 116 (depicted as arrows 160) directly to the internal tissue 150 (i.e., without first being transmitted through another tissue), and directly from the internal tissue 150 to the detector 26, 118 (i.e., without being transmitted through another tissue).

Again, as noted above, such a configuration may result in enhanced light transmission efficiency, which may reduce power consumption by the emitter 24 compared to configurations in which light is transmitted through the skin layer 150. Furthermore, the values for the monitored physiological parameter obtained from light detected by the detector 26, 118 are more likely to be representative of the internal tissue 150 rather than the entire region encompassing the internal tissue 150. Such location-specific information may be desirable to ascertain the condition of the internal tissue 150, for example after a surgical or interventional procedure where healing or other indicators of tissue health, such as indications of restored blood flow, may be desired. For example, the internal tissue 150 may be the dermis or subcutis of the patient's skin, or may be an internal organ such as a kidney, a liver, a heart, vasculature, muscle, or any other internal tissue for which light-based monitoring may be suitable.

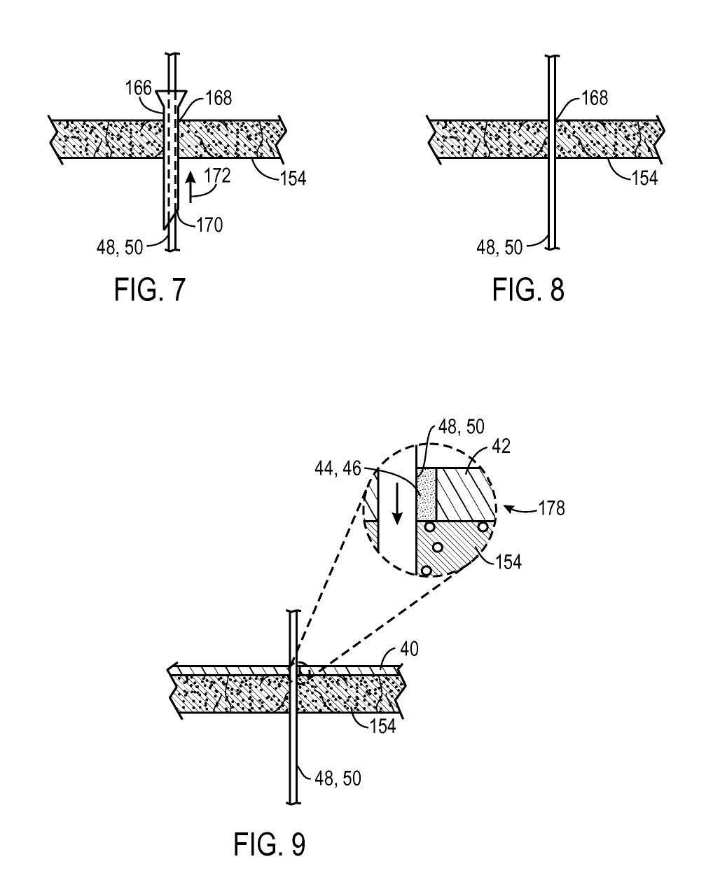

As discussed above, the first and second light guides 48, 50 are implantable to enable monitoring in the manner discussed above. FIG. 6 depicts a process flow diagram of an embodiment of a method 160 for placing the first and/or second light guides 48, 50 below a skin layer. To facilitate description of certain of the steps included in the method 160, reference is also made to FIGS. 7-9, which schematically depict the configuration obtained from performing the steps on the patient. The method 160, in the embodiment of FIG. 6, includes inserting (block 162) a needle or similar guide into a region of interest of the patient. As an example, a needle may be inserted through the skin of the patient, such as through an outer skin layer (e.g., the epidermis), or through additional skin layers (e.g., the dermis and hypodermis). Accordingly, the patient's skin is penetrated to a desired depth. In accordance with an embodiment, the acts of block 162 may be performed in conjunction with an imaging system, such as an ultrasound system or an X-ray fluoroscopic imaging system, so as to enable the healthcare practitioner to monitor the position of the needle relative to the internal tissue of interest. Additionally or alternatively, the length of the needle may be measured before skin penetration and after skin penetration to determine the depth to which the needle has been inserted into the patient.

After the needle has been suitably positioned, the light guide (e.g., first and/or second light guides 48, 50) may be threaded (block 164) through the needle. For example, referring to FIG. 7, one embodiment of a configuration resulting from the acts of blocks 162 and 164 is depicted. As illustrated, a needle 166 has penetrated the skin layer 154, forming an opening 168 in the skin layer 154 in which the first and/or second light guides 48, 50 are positioned. Specifically, the first or second light guides 48, 50, are threaded through a central opening 170 of the needle 166, which is disposed in the opening 168. The combined assembly of the needle 166 and the first or second light guides 48, 50 may be re-positioned as desired to obtain the desired positioning relative to, for example, the internal tissue 150 (FIG. 5) for the first or second light guides 48, 50.

As also shown by an upward arrow 172 in FIG. 7, returning to the method 160 of FIG. 6, once the first and/or second light guides 48, 50 have been positioned as desired, the needle 166 may be retracted (block 174). The resulting configuration after retraction of the needle 166 according to block 174 is depicted in FIG. 8, in which the needle 166 is no longer present, and the first and/or second light guides 48, 50 occupy the space defined by the opening 168 in the skin 154. In certain embodiments, the size (e.g., a diameter) of the first and/or second light guides 48, 50 may be such that the patient is not disturbed by the presence of the light guides 48, 50.

Returning again to FIG. 6, the method 160 also includes placing (block 176) the bandage 40 over the region of interest and over the first and/or second light guides 48, 50. For example, the acts according to block 176 may include aligning the first and/or second light guides 48, 50 with the emitter and detector lens portions 44, 46 of the bandage 40 (FIG. 5), and securing the bandage 40 to the patient's skin (e.g., skin layer 154). The resulting configuration is depicted in FIG. 9. As illustrated in FIG. 9, the bandage 40 is in physical contact with the skin layer 154, and may be attached via an adhesive as discussed above.

Further, as depicted in expanded region 178, the first and/or second light guide 48, 50 is illustrated as positioned within or otherwise coupled to the corresponding emitter and/or detector lens portion 44, 46. The first and/or second light guide 48, 50 may be coupled to the corresponding emitter and/or detector lens portion 44, 46 using any suitable coupling mechanism, including a biodegradable or non-biodegradable adhesive, or using a small amount of a suspension or solution of a biodegradable material, such as fibroin, poly(lactic acids), synthetic and natural silks, cellulose and its derivatives, chitin and chitosan derivatives, alginates, sugars, and poly(hydroxyalkanoates), or any combination thereof. In such embodiments, the first and/or second light guide 48, 50 may be coupled to the corresponding emitter and/or detector lens portion 44, 46 by disposing an amount of the solution into the emitter and/or detector lens portion 44, 46, disposing the first and/or second light guides 48, 50 therein, and drying the solution such that the resulting dried biodegradable material couples the first and/or second light guide 48, 50 to the corresponding emitter and/or detector lens portion 44, 46. Once the bandage 40 is suitably positioned on the patient, the sensor 14, 112 may be coupled (block 180) thereto in the manner discussed above with respect to FIG. 5.

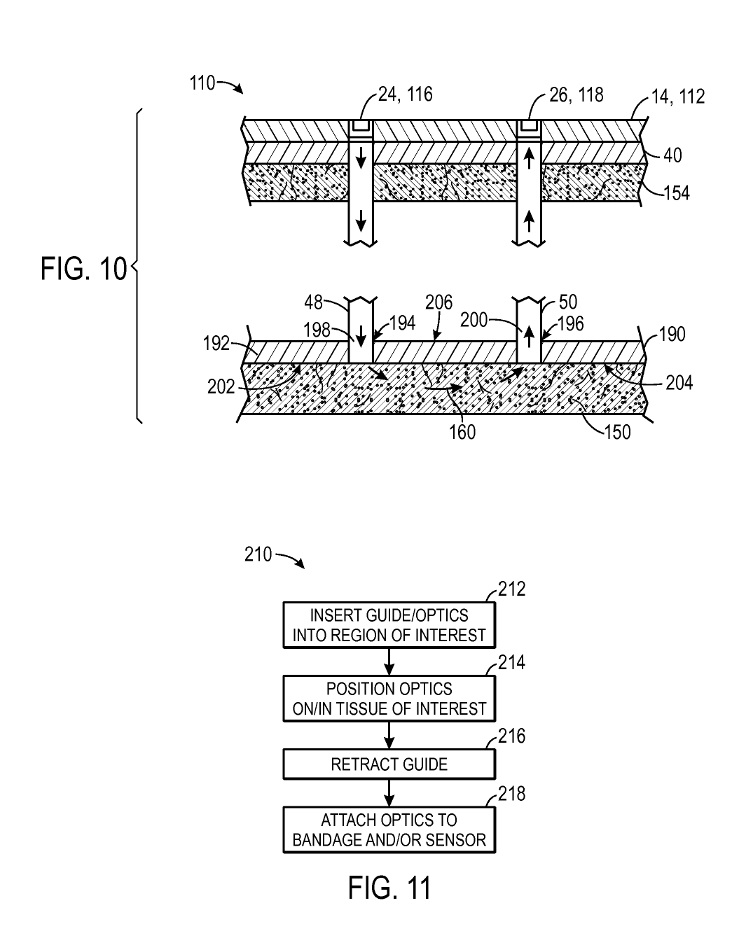

While certain of the embodiments described above relate to the positioning of light guides against the internal tissue 150 of the patient, in some situations it may be desirable to use the bandage 40 and its associated optical features in conjunction with an additional, implantable bandage. Such a configuration is depicted as a cross-sectional diagram in FIG. 10. In particular, FIG. 10 includes the sensor assembly 110 discussed above with respect to FIG. 5, and includes an additional bandage 190 secured or otherwise in physical contact with the internal tissue 150. In other words, in the illustrated configuration, the bandage 40 of the sensor assembly 110 is a first bandage, and the additional bandage 190 is a second bandage, where the first bandage (i.e., bandage 40) is positioned against the patient's skin layer 154 and the second bandage (i.e., additional bandage 190) is positioned against the internal tissue 150.

As depicted, the additional bandage 190 includes a body 192 having first and second receiving portions 194, 196 for the first and second light guides 48, 50, respectively. Specifically, the first and second receiving portions 194, 196 may receive respective ends 198, 200 of the first and second light guides 48, 50 so as to position the ends 198, 200 directly against the internal tissue 150, or a desired distance away from the internal tissue 150 suitable for performing the desired physiological measurements. Indeed, the first and second receiving portions 194, 196 may include a suitable coupling mechanism for coupling the first and second light guides 48, 50 to the additional bandage in a similar manner as discussed above with respect to FIG. 9. That is, the first and second receiving portions 194, 196 may couple to the first and second light guides 48, 50 using a biodegradable or non-biodegradable adhesive, or using a small amount of a suspension or solution of a biodegradable material, such as fibroin, poly(lactic acids), synthetic and natural silks, cellulose and its derivatives, chitin and chitosan derivatives, alginates, sugars, and poly(hydroxyalkanoates), or any combination thereof. In such embodiments, the first and second light guides 48, 50 may be coupled to the corresponding first or second receiving portions 194, 196 by disposing an amount of the solution into the first and second receiving portions 194, 196, disposing the first and second light guides 48, 50 therein, and drying the solution such that the resulting dried biodegradable material couples the first and/or second light guide 48, 50 to the corresponding first and second receiving portions 194, 196.

The additional bandage 190 and, more specifically, the body 192 and first and second receiving portions 194, 196 of the additional bandage 190, may be constructed using biodegradable or non-biodegradable materials. By way of non-limiting example, any one or a combination of the body 192 and first and second receiving portions 194, 196 of the additional bandage 190 may be constructed from a biodegradable material such as fibroin isolated from silkworm silk, poly(lactic acids), synthetic and natural silks, cellulose and its derivatives, chitin and chitosan derivatives, alginates, sugars, and poly(hydroxyalkanoates), or any combination thereof. Further, because the additional bandage 190 has the benefit of being positioned directly against the patient's internal tissue 150, one or more active agents (e.g., therapeutic agents) may be incorporated into its construction to facilitate healing, patient comfort, or the like.

For example, during use, the additional bandage 190 may be utilized to both monitor one or more physiological parameters of the patient's internal tissue 150 and to provide a steady source of an active agent to the internal tissue 150. Further, the additional bandage 190 may be constructed from suitable materials such that as it degrades in the patient's body, the additional bandage 190 also controllably releases a therapeutic agent to the internal tissue 150. Indeed, the patient monitor 12 (FIGS. 1 and 2) may monitor the signals generated by the detector 26, 118 for feedback indicative of degradation of the additional bandage 190. For example, the feedback may include greater than normal noise in the signal (e.g., due to shunting), or a low average signal compared to when the additional bandage 190 is first positioned within the patient. Such feedback may also indicate that it is time to change the additional bandage 190 because the body 192 having the active agent may be substantially depleted of the active agent.

As discussed above, during operation, the emitter 24, 116 emits light, which is transmitted along the first light guide 48. In the illustrated embodiment, the light also traverses the additional bandage 190 via the first receiving portion 194, and is transmitted through the tissue (as depicted by arrows 160). The transmitted light is then received at the second receiving portion 196 and into the second light guide 50, which transmits this light to the detector 26, 118. It should be noted that the body 192 of the additional bandage 190, while illustrated as being constructed from one material, may have a similar construction as set forth above with respect to the bandage 40 in FIG. 4. In other words, the body 192 of the additional bandage 190 may have an emitter region 202, a detector region 204, and a light barrier region 206 between its respective emitter and detector regions 202, 204. As noted above with respect to FIG. 4, it may be desirable to have the emitter and detector regions 202, 204 be transparent to the light so as to maximize the monitored area of the internal tissue 150, while preventing light shunting using the light barrier region 206. On the other hand, in certain embodiments, it may be desirable to obtain information related to a very particular portion of the internal tissue 150, such as an implant, suture, or the like. In such embodiments, the emitter and/or detector regions 202, 204 may be opaque to the light emitted by the emitter 24, 116 such that the light is only received at the second receiving portion 196. Accordingly, the emitter and detector regions 202, 204 may independently be transparent or opaque to the light emitted by the emitter 24, 116.

Similarly, the size of the additional bandage 190 may also affect the area of the internal tissue 150 that is monitored. For example, relatively large sizes for the additional bandage 190 may result in larger monitored areas of the internal tissue 150, while smaller sizes result in smaller monitored areas. Indeed, the additional bandage 190 may be smaller, larger, or substantially the same size as the bandage 40. For example, in embodiments where the additional bandage 190 is smaller than the bandage 40, the additional bandage 190 may have an overall surface area that is less than approximately 99% of the overall surface area of the bandage 40, such as between approximately 1% and 95%, 10% and 90%, 30% and 70%, or approximately 50% of the overall surface area of the bandage 40. Such embodiments of the additional bandage 190 may be desirable to facilitate biodegradation, or to monitor specific areas of the internal tissue 150 (e.g., a wound or post-surgical closure). Conversely, in embodiments where the bandage 40 is smaller than the additional bandage 190 (i.e., the additional bandage 190 is larger than the bandage 40), the bandage 40 may have an overall surface area that is less than approximately 99% of the overall surface area of the additional bandage 190, such as between approximately 1% and 95%, 10% and 90%, 30% and 70%, or approximately 50% of the overall surface area of the additional bandage 190. Such embodiments may be desirable to enable monitoring of a larger area of the internal tissue 150, to enable extended periods of use, or the like.

Indeed, using any one or a combination of the arrangements described above (e.g., the sensor 14 or 116 in combination with either or both of the bandage 40 and additional bandage 190), a caregiver may monitor the internal tissue 150 of the patient for a relatively extended period of time in a relatively cost-effective manner. For example, such monitoring may reduce the reliance on expensive imaging techniques by providing location-specific physiological information, which may be indicative of healing, a lack of healing or recovery, or similar situation.

While certain tissues may be readily accessible via a hypodermic needle as discussed above, or using a small incision, or even during major surgical procedures (e.g., heart surgery) while a portion of the body cavity is open, in some situations, such as with deep internal tissues and when a major surgical procedure is not being performed, are not as accessible. Accordingly, it may be desirable to use a guide (e.g., a catheter or other endoscopic device) to place the light guides 48, 50 and/or the additional bandage 190 in place. FIG. 11 is a process flow diagram depicting an embodiment of a method 210 for placing the desired features in this manner.

As illustrated, the method 210 includes inserting the guide, along with the desired optics, into a region of interest (block 212). Again, the guide may be a catheter or similar endoscopic device, and the optics may include the light guides 48, 50, the additional bandage 190, all or a portion of light emitting and light detecting features of the sensor 14, 112, or any combination thereof. In addition, the region of interest into which the guide and associated optics are inserted may or may not correspond to the area desired for monitoring. As one example, the guide may be a catheter associated with the light guides 48, 50 and/or the additional bandage 190, and may be inserted into a leg of the patient. The guide may be run through the vasculature of the patient (e.g., via the iliac or femoral artery or vein). The guide may then be moved through the vasculature (e.g., through the aorta or the inferior vena cava) and to a tissue of interest. For example, in embodiments where the tissue of interest is the kidney, the catheter may be run through the renal artery or vein. In embodiments where the heart may be monitored, the guide may be run through the vena cava or aorta.

After reaching the tissue of interest, the optics may be positioned in or on the tissue of interest, for example via insertion into the tissue 150 or a simple placement against the exterior of the tissue 150 (block 214). Once the desired optics are in the desired position, the guide may then be retracted (block 216). For example, the optics may be inserted, adhered to, or otherwise positioned against the tissue of interest, and the guide may detach from the optics, facilitating retraction. In other embodiments, however, the guide may not necessarily be retracted immediately (e.g., during a surgical procedure). Indeed, the optics may be used in conjunction with the guide, such as when the guide is an endoscopic or similar device, to provide physiological information about the internal tissue 150 as the internal tissue 150 is viewed/treated. In other words, in certain embodiments, the acts of block 216 may not necessarily be performed before monitoring is performed.

Before, during, or after the optics (e.g., light guides 48, 50, additional bandage 190) are in place, the optics may be attached to the bandage 40 and/or the sensor 14, 112 (block 218). For example, in embodiments where the optics do not include the bandage 40 (i.e., the bandage that attaches to the skin layer 154), the light guides 48, 50 may be attached to the emitter and detector lens portions 44, 46, and the bandage 40 may be attached to the sensor 14, 112. In one embodiment, the sensor 14, 112 and the bandage 40 may already be attached to one another.

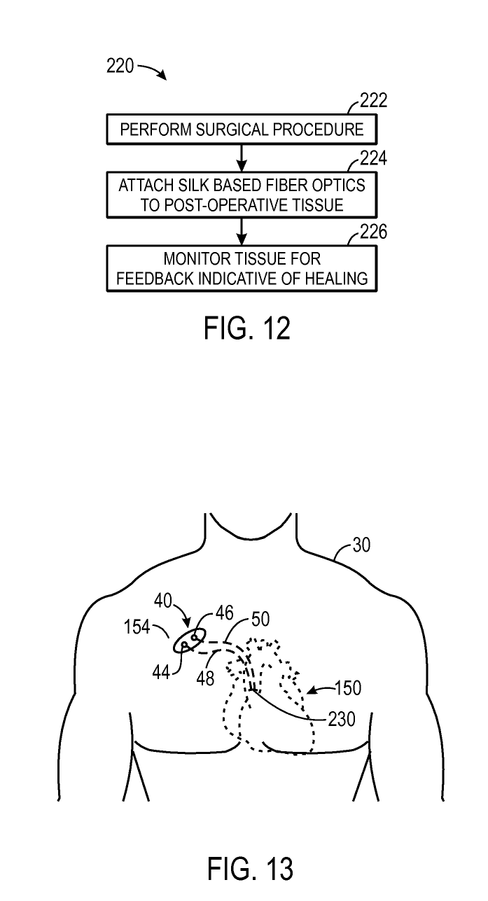

As noted above, during certain situations, such as during a surgical procedure, it may be desirable to monitor one or more physiological parameters related to that particular tissue, or to another tissue that may be affected during the procedure. FIG. 12 is a process flow diagram depicting such an embodiment of a method 220 for monitoring post-operative tissue, though the particular tissue that is monitored is not necessarily limited to the tissue on which the procedure is being performed. The method 220 includes performing a surgical procedure on a patient tissue (block 222). The surgical procedure may include any interventional, non-invasive, or invasive procedure in which a patient's tissue may be treated, biopsied, ablated, punctured, incised, modified, reshaped, or the like. Generally, any procedure in which tissue healing is desired, or in which the tissue's proper function is in question, is presently contemplated.

Once the surgical procedure is performed in accordance with block 222, biodegradable optical elements in accordance with present embodiments are attached to the post-operative tissue (block 224). For example, the first and second light guides 48, 50, and/or the additional bandage 190 may be attached to the internal tissue 150. The attachment may be rigid, such as by inserting the first and second light guides 48, 50 into the internal tissue 150, or by adhering the additional bandage 190 to the internal tissue 150, or a combination thereof. For example, the additional bandage 190 may be adhered to the internal tissue 150 via a biodegradable or biocompatible adhesive. In other embodiments, the additional bandage 190 may simply be placed into contact with the internal tissue 150 and held in place by adherence caused by the moisture of the tissue. In certain embodiments, the acts according to block 224 may be performed according to the method 160 described above with respect to FIGS. 6-9, or the method 210 discussed above with respect to FIG. 11.

Regardless of the particular method used for placement, once the desired monitoring structures are in place (e.g., including the sensor 14, 112), the sensor 14, 112 and the patient monitor 12 are utilized to monitor the patient's tissue (e.g., the internal tissue 150) for feedback indicative of healing (block 226). While such feedback will generally include SpO₂ data when the sensor 14, 112 is a pulse oximetry sensor, other types of feedback may also be determined, such as the patient's glucose levels, hemoglobin levels, hematocrit levels, tissue hydration, as well as other physiological parameters.

It should be noted that a variety of tissues may be monitored according to the methods disclosed herein, including the method 220 of FIG. 12. Indeed, tissues including the heart, liver, kidneys, musculature, and the like, are presently contemplated, as discussed above. The particular type of tissue being monitored may, at least partially, dictate the manner in which the sensing features (e.g., the light guides 48, 50, the additional bandage 190) are placed against the monitored tissue. In addition, the nature of the particular surgical procedure, such as whether the procedure is a minimally invasive interventional procedure or a highly invasive major surgical procedure (e.g., heart surgery), may also be a factor.

Indeed, while the steps of the method 220 of FIG. 12 may be performed at separate and distinct stages of procedure and recovery, in some embodiments, they may be performed in conjunction with one another. For example, in some procedures, the optics used for monitoring may be positioned during the surgical procedure such that the acts of blocks 222 and 224 are performed in conjunction with one another. In such embodiments, the acts of block 224 may include attaching the optics to pre-operative tissue, intra-operative tissue, or post-operative tissue as illustrated. Further, the monitoring acts of block 226 may also be performed in conjunction with the surgical procedure to obtain intra-operative physiological information regarding the particular tissue. By way of example, before, during, or after a major surgical procedure, while the tissue is still readily accessible by the caregiver, the caregiver (e.g., a surgeon) may position the light guides 48, 50 and/or the additional bandage 190 within or against the internal tissue 150. One such embodiment is depicted schematically in FIG. 13.

In particular, FIG. 13 depicts an arrangement in which the first and second light guides 48, 50 have been positioned within, or immediately adjacent to, an area 230 of the internal tissue 150 (in this embodiment, the heart) where a surgical procedure has been performed. In the illustrated embodiment, the light guides 48, 50 terminate at one end at the bandage 40, which is adhered to the skin layer 154 (e.g., the epidermis) to facilitate external attachment to the sensor 14, 112. While illustrated as being positioned away from the region in which the heart is located to facilitate discussion, the bandage 40 may actually be positioned directly over the incision used during the surgical procedure to reach the heart (e.g., the incision's closure), such that the light guides 48, 50 may traverse the skin layer 154 and attach to the bandage 40 without additional incisions, punctures, or the like.

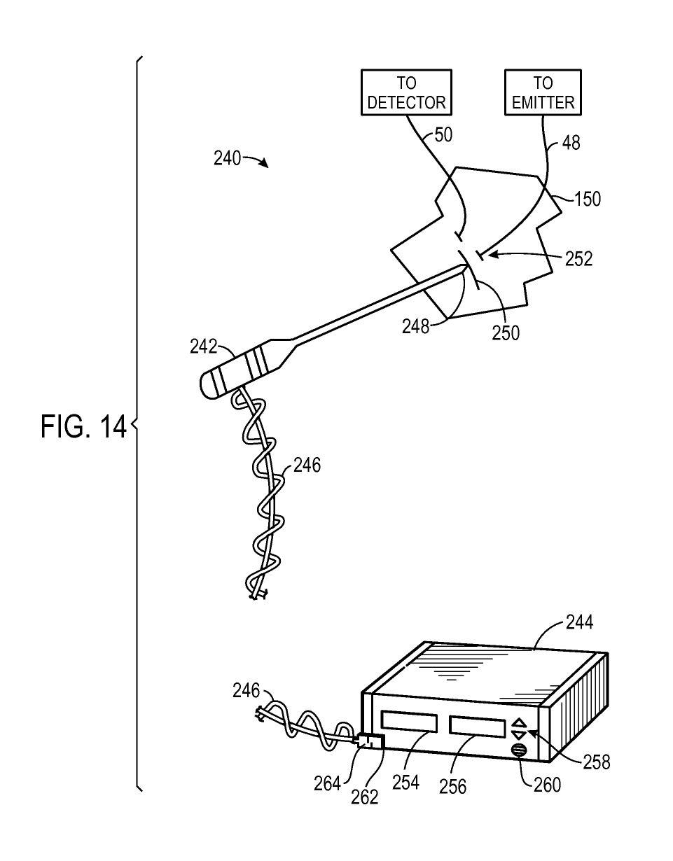

In some situations, as noted above, it may be desirable to monitor tissue health during a surgical procedure, such as to provide additional feedback during the procedure in addition to the feedback mechanisms already in place. Again, the procedure may be an interventional one, a major surgical procedure, or the like. In one embodiment, depicted schematically in FIG. 14, the procedure may be performed using a surgical system 240, which includes a surgical tool 242 and a surgical control system 244 operatively coupled to the surgical tool 242 via one or more cables 246. By way of non-limiting example, the surgical tool 242 may be an electrosurgical device, a microwave ablation device, a radiofrequency ablation device, or any similar device. In particular, the surgical system 240 may be an EVIDENT™ microwave ablation system or a FORCETRIAD™ energy platform system, and the surgical tool 242 may be a FORCE TRIVERSE™ electrosurgical tool, a LIGA SURE™ tissue fusion tool, an EVIDENT™ microwave ablation percutaneous/laparoscopic antenna, or an EVIDENT™ microwave ablation open surgical antenna, all of which are available from Covidien of Mansfield, Mass.

Therefore, in a general sense, the surgical tool 242 utilizes (e.g., deposits) energy into the internal tissue 150 to cause ablation, fusion, cutting, or the like. In certain embodiments, the surgical system 240 may also include other elements such as a patient return electrode pad, or the like. Other sensing elements, such as electrodes, may provide feedback to the surgical control system 244, and the surgical control system 244 may adjust the power or energy provided to the surgical tool 242 as a result of the feedback. One example of such feedback is tissue impedance, which may be used in conjunction with radiofrequency ablation systems.

In accordance with present embodiments, feedback relating to the perfusion, hydration, or other similar physiological parameters of the internal tissue 150 may also be provided to the surgical control system 244. For example, in the illustrated embodiment, the surgical tool 242 includes an active tip 248, which is used to deposit energy into the internal tissue 150 to cause an operative area 250 to be opened, fused, or the like. The first and second light guides 48, 50 may be positioned in a region 252 proximate the operative area 250 to provide feedback related to one or more physiological parameters in the region 252. Such feedback may be desirable to ensure that the proper amount of energy is being deposited into the internal tissue 150 for the desired size of the operative area 250. In other words, in situations where a suitable amount of energy is provided to the operative area 250 to obtain a desired size for the area 250, the feedback for the region 252 may be indicative of normal tissue health. On the other hand, in situations where the energy being provided to the operative area 250 is too high, the feedback obtained from the region 252 may indicate that too much energy is being deposited into the internal tissue 150 to obtain the desired size for the operative area 250.

In certain embodiments, the internal tissue 150 may, for example, be a cancerous portion of a tissue being treated. In such embodiments, the surgical system 240 may be used to ablate the internal tissue 150 at a size encompassing the monitored region 252. Accordingly, the region 252 may be monitored for feedback indicative of tissue death, such as reduced perfusion or hydration levels.

The feedback described herein may be provided directly to the surgical control system 244, which may cause the surgical control system 244 to automatically adjust, or the feedback may be provided, for example, via the monitor 12 of FIG. 1 to a caregiver, who may then adjust various parameters on the surgical control system 244. Indeed, as illustrated, the surgical control system 244 includes features that enable the caregiver to view and adjust one or more operational parameters of the surgical system 240, including one or more displays 254, 256 configured to display information relating to the feedback (e.g., SpO₂, tissue impedance) obtained from one or more sensing features (e.g., sensor 14, 112), and to display information relating to the operation of the surgical system 240. The surgical control system also includes one or more controls 258 for adjusting various operational parameters of the system 240, such as the power/energy provided to the surgical tool 242, time limits for the operation of the surgical tool 242, to switch operational modes of the surgical tool 242 (e.g., between electrosurgical cutting, coagulating, or ablating), or similar operating parameters. The surgical control system 244 may also include a speaker 260 for providing audible feedback to a caregiver or technician relating to the state of the surgical tool 242, the surgical control system 244, or the physiological parameters being monitored. Indeed, in certain embodiments, the surgical control system 244 may interface with one or more patient monitors (e.g., patient monitor 12) to enable a caregiver to obtain information from a centralized location, and/or to facilitate adjusting operating parameters of the surgical system 240 in an automatic and expedient manner.

It should be noted that the surgical control system 244 may include a processor-based machine capable of receiving data, monitoring/processing the data, and performing various control actions based on this monitoring/processing. For example, the surgical control system 244 may include one or more non-transitory, tangible, machine-readable media collectively storing instructions for performing the various monitoring and control actions described herein. The surgical control system 244 may also include one or more processors for executing the instructions to perform the acts discussed below. Indeed, the surgical control system 244 may further include features for communicating with the surgical tool 242, such as a connector 262 suitable for receiving a cable connector 264 of the surgical tool 242. Electrical energy for driving the surgical tool 242, or suitable microwave, radiofrequency, or other energy, may also be provided via the connector 262 to the surgical tool 242 for performing various surgical procedures. The surgical control system 244 may also receive feedback from the surgical tool 242, including operating parameters, measured physiological parameters (e.g., tissue impedance), or the like, via the connector 262. Indeed, the surgical control system 244 may include any features suitable for enabling the surgical system 240 to perform the monitoring, control, and surgical acts described herein.

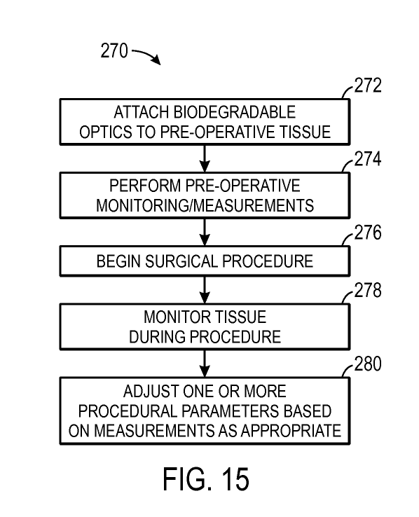

As noted above, the internal tissue 150 may be monitored during a surgical procedure, such as a procedure performed using the surgical system 240. In certain embodiments, a caregiver may observe values of monitored physiological parameters, and may adjust a procedure based on the monitored parameters. In other embodiments, however, this may be performed, at least partially, by a system having the surgical equipment being used by the caregiver (e.g., the surgical system 240). One embodiment of a method 270 in which physiological parameters are monitored using the biodegradable optical features described herein during a surgical procedure is depicted as a process flow diagram in FIG. 15.

As illustrated, the method 270 includes attaching biodegradable optics (e.g., the first and second light guides 48, 50, the additional bandage 190) to a pre-operative tissue (block 272) in any suitable manner, such as using the methods 160 or 210 discussed above with respect to FIGS. 6 and 11, respectively. Again, the tissue is not limited to the tissue on which the surgery is being performed. In one embodiment, the tissue is the tissue on which the surgery is being performed. Once optics are in place, and are suitably attached to a sensing device (e.g., sensor 14, 112), pre-operative measurements of the internal tissue 150 may be obtained (block 274). For example, it may be desirable to obtain such measurements to establish baseline values for various physiological parameters of interest for later comparison during the surgical procedure.

Having suitably established baseline values, the surgical procedure may be performed (block 276). As discussed above with respect to FIG. 12, any surgical procedure in which monitoring may be beneficial is presently contemplated. Such surgical procedures include, but are not limited to, incision, ablation, coagulation, tissue fusion, ligation, and so on.

During the surgical procedure, various measurement values for one or more physiological parameters are obtained (block 278) using at least the optics (e.g., the first and second light guides 48, 50, the additional bandage 190) attached to the internal tissue 150. In one embodiment, the values obtained are SpO₂ values.

As the tissue is monitored, the caregiver, the surgical system (e.g., the surgical system 240), such as a controller of the surgical system (e.g., the surgical control system 244), or any combination thereof, may adjust (block 280) one or more operating parameters of the procedure. This adjustment may simply include the caregiver (e.g., a surgeon) re-positioning a catheter or an electrosurgical device, or may include adjusting an intensity of energy being deposited on the tissue being treated. In one embodiment, the surgical system (e.g., the surgical control system 244) may automatically determine, based at least partially on the physiological parameters measured using the optics, that one or more operating parameters may be suitable for adjustment. For example, the surgical control system 242 may provide an audible, visual, or tactile indication (e.g., via the speaker 260, the displays 254, 256, or the surgical tool 242) to the caregiver to adjust the one or more operating parameters, or may automatically adjust the parameters.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A physiological sensor assembly, comprising:
a physiological sensor, comprising:
a sensor body;
an emitter disposed in the sensor body and configured to emit one or more wavelengths of light toward a patient; and
a detector disposed in the sensor body and configured to detect the one or more wavelengths of light after transmission through a tissue of the patient;
a first biodegradable light guide configured to be placed in optical communication with the emitter, wherein the first biodegradable light guide is configured to physically traverse a skin layer of the patient to transmit the one or more wavelengths of light emitted by the emitter to an internal tissue of the patient; and
a second biodegradable light guide configured to be placed in optical communication with the detector, wherein the second light guide is configured to physically traverse the skin layer of the patient to transmit the one or more wavelengths of light transmitted by the emitter through the internal tissue to the detector to measure a physiological parameter of the internal tissue.

2. The physiological sensor assembly of claim 1, wherein the first biodegradable light guide comprises a first geometry that enables the first biodegradable light guide to pierce the skin layer of the patient, and the second biodegradable light guide comprises a second geometry that enables the second biodegradable light guide to pierce the skin layer of the patient.

3. The physiological sensor assembly of claim 2, comprising a first plurality of biodegradable microneedles positioned on a lens of the emitter and a second plurality of biodegradable microneedles positioned on a lens of the detector, wherein the first plurality of biodegradable microneedles comprises the first biodegradable light guide, and the second plurality of biodegradable microneedles comprises the second biodegradable light guide.

4. The physiological sensor assembly of claim 3, wherein each microneedle of the first and second plurality of microneedles has a dimension between approximately 10 nm and 50 μm.

5. The physiological sensor assembly of claim 1, comprising:
a first bandage, comprising:
a first bandage body;
a first emitter opening disposed in the first bandage body and configured to optically couple the emitter of the physiological sensor with the first biodegradable light guide; and
a first detector opening disposed in the first bandage body and configured to optically couple the detector of the physiological sensor with the second biodegradable light guide; and
wherein the first bandage is configured to be placed in physical contact with the skin layer of the patient on a patient-contacting side of the first bandage body, and the first bandage is configured to be coupled with the physiological sensor on a non-patient-contacting side of the first bandage body.

6. The physiological sensor assembly of claim 5, wherein the first bandage body comprises a first emitter region positioned about the first emitter opening and a first detector region positioned about the first detector opening, and the first emitter and detector regions are independently transparent or opaque to the one or more wavelengths of light emitted by the emitter.

7. The physiological sensor assembly of claim 5, wherein the first bandage body comprises a coupling mechanism configured to secure the first bandage body to the physiological sensor, wherein the coupling mechanism comprises an adhesive, a hook-and-loop connector, or a combination thereof.

8. The physiological sensor assembly of claim 5, comprising:
a second bandage, comprising:
a second bandage body;
a second emitter opening disposed in the second bandage body and configured to optically couple the first biodegradable light guide with the internal tissue of the patient; and
a second detector opening disposed in the second bandage body and configured to optically couple the second biodegradable light guide with the internal tissue of the patient; and
wherein the second bandage is configured to be placed against the internal tissue of the patient on a patient-contacting side of the second bandage body.

9. The physiological sensor assembly of claim 8, wherein at least a portion of the first body of the first bandage, at least a portion of the second body of the second bandage, or a combination thereof, comprise one or more biodegradable materials.

10. The physiological sensor assembly of claim 8, wherein at least a portion of the first body of the first bandage, at least a portion of the second body of the second bandage, the first biodegradable light guide, the second biodegradable light guide, or a combination thereof, comprise fibroin isolated from silkworm silk.

11. The physiological sensor assembly of claim 8, comprising a biodegradable adhesive disposed on the patient-contacting side of the second bandage body, wherein the adhesive is configured to secure the second bandage body to the internal tissue.

12. The physiological sensor assembly of claim 1, wherein the physiological sensor comprises a pulse oximetry sensor.

13. A patient monitoring system, comprising:
a physiological sensor assembly, comprising:
a physiological sensor, comprising:
a sensor body;
an emitter disposed in the sensor body and configured to emit one or more wavelengths of light toward a patient; and
a detector disposed in the sensor body and configured to detect the one or more wavelengths of light after transmission through a tissue of the patient; and
one or more biodegradable light guides configured to be placed in optical communication with the emitter, the detector, or a combination thereof, wherein the one or more biodegradable light guides are configured to physically traverse a skin layer of the patient to transmit the one or more wavelengths of light emitted by the emitter to an internal tissue of the patient, to transmit the one or more wavelengths of light transmitted by the emitter through the internal tissue to the detector, or a combination thereof, to measure a physiological parameter of the internal tissue; and
a physiological monitor configured to receive data from the physiological sensor to monitor the physiological parameter of the internal tissue.

14. The patient monitoring system of claim 13, wherein the one or more biodegradable light guides each comprise a geometry that enables the respective biodegradable light guide to pierce the skin layer of the patient.

15. The patient monitoring system of claim 13, comprising:
a first bandage comprising a first bandage body having one or more first openings, wherein the one or more first openings are configured to optically couple the one or more biodegradable light guides with the emitter, the detector, or a combination thereof; and
wherein the first bandage is configured to be placed in physical contact with the skin layer of the patient on a patient-contacting side of the first bandage body, and the first bandage is configured to be coupled with the physiological sensor on a non-patient-contacting side of the first bandage body.

16. The patient monitoring system of claim 15, comprising:
a second bandage comprising a second bandage body having one or more second openings, wherein the one or more second openings are configured to secure the one or more biodegradable light guides relative to the internal tissue of the patient; and
wherein the one or more biodegradable light guides are configured to transmit the one or more wavelengths emitted by the emitter into the internal tissue, or to transmit the one or more wavelengths transmitted through the internal tissue to the detector, or a combination thereof.

17. A medical system, comprising:
a physiological sensor assembly configured to obtain physiological data of an internal tissue, wherein the physiological sensor assembly comprises:
an emitter configured to emit one or more wavelengths of light toward the internal tissue;
a detector configured to detect the one or more wavelengths of light after transmission through the internal tissue; and
one or more biodegradable light guides configured to be placed in optical communication with the emitter, the detector, or a combination thereof, wherein the one or more biodegradable light guides are configured to physically traverse a skin layer of the patient to optically couple the emitter, the detector, or a combination thereof, to the internal tissue; and
a surgical system configured to receive feedback indicative of the physiological data generated by the physiological sensor assembly, wherein the surgical system comprises:
a surgical tool configured to enable a caregiver to perform a surgical technique on the internal tissue; and
a surgical control system configured to control one or more operational parameters of the surgical tool in response to the physiological data.

18. The medical system of claim 17, comprising a physiological monitor configured to receive the physiological data from the physiological sensor assembly to monitor a physiological parameter of the internal tissue, wherein the physiological monitor generates the feedback provided to the surgical system.

19. The medical system of claim 17, wherein the physiological sensor assembly comprises:
a first bandage comprising a first bandage body having one or more first openings, wherein the one or more first openings are configured to optically couple the one or more biodegradable light guides with the emitter, the detector, or a combination thereof; and
wherein the first bandage is configured to be placed in physical contact with the skin layer of the patient on a patient-contacting side of the first bandage body, and the first bandage is configured to be coupled with a physiological sensor having the emitter and the detector on a non-patient-contacting side of the first bandage body.

20. The medical system of claim 17, wherein the surgical tool is an electrosurgical device, a microwave ablation device, a radiofrequency ablation device, or any combination thereof, and the physiological sensor assembly comprises a pulse oximetry sensor having at least the emitter and the detector.
